(19) United States
(12) Patent Application Publication
(10) Pub. No.: US 2024/0410877 A1
(43) Pub. Date: Dec. 12, 2024

(54) SYSTEMS AND METHODS FOR MIXED MULTIPLE CELL LINE SCREENING USING ENDOGENOUS SINGLE NUCLEOTIDE POLYMORPHISM (SNP)-BASED CELL LINE IDENTIFICATION

(71) Applicant: University of Kentucky Research Foundation, Lexington, KY (US)

(72) Inventors: Chunming Liu, Lexington, KY (US); Chi Wang, Lexington, KY (US); Shulin Zhang, Lexington, KY (US)

(21) Appl. No.: 18/740,914

(22) Filed: Jun. 12, 2024

Related U.S. Application Data
(60) Provisional application No. 63/472,478, filed on Jun. 12, 2023.

(51) Int. Cl. G01N 33/50

(52) U.S. Cl. CPC G01N 33/5023

(57) ABSTRACT

Methods for screening a test compound against multiple cell lines are provided and include the steps of: treating a mixture including cells from multiple cell lines with the test compound; identifying allele frequencies of single nucleotide polymorphisms (SNPs) in the treated mixture and a control mixture including cells from the same cell lines as present in the treated mixture; estimating a proportion of each individual cell line in both the treated mixture and the control mixture; and quantifying the effect of the test compound on each individual cell line using the estimated proportion of each cell line in the treated mixture and the estimated proportion of each cell line in the control mixture. Systems for screening a test compound against multiple cell lines, as well as systems and methods for identifying a proportion of a particular cell line in a mixture including multiple cell lines, are also provided.

### RELATED APPLICATIONS

The present application claims priority to U.S. Patent Application Ser. No. 63/472,478, filed on Jun. 12, 2023, the entire disclosure of which is incorporated herein by reference.

### TECHNICAL FIELD

The presently disclosed subject matter generally relates to the screening of test compounds against cell lines to assess the effect thereof. In particular, certain embodiments of the presently disclosed subject matter relate to methods and systems for cell line screening that make use of endogenous single nucleotide polymorphisms (SNPs) to assess the efficacy of a test compound against multiple cell lines.

### BACKGROUND

Drug development is a long and expensive process. Cell line screening is often performed before testing a drug candidate in animal models for its in vivo efficacy, pharmacology and toxicity. Indeed, cancer cell line screening, for example, is an important tool for anti-cancer drug development.

High throughput screening methods have been established by a number of institutions and companies. For example, the National Cancer Institute (NCI) has established an NCI60 Human Tumor Cell Lines Screen program, which collected 60 different human cancer cell lines and provided screening service for drug development and research. This is one of the first high throughput programs for drug screening in multiple cancer cell lines. Over 10,000 compounds have been screened through NCI60 since 1989. Based on compound information for a candidate drug and the screening results, a computer program called COMPARE was developed. The algorithm of the COMPARE computer program can compare the patterns of a test compound (candidate drug) with other compounds in the database, thus revealing potential mechanisms for the test compound.

For another example of an established high-throughput cell screening platform, the Broad Institute published a Cancer Cell Line Encyclopedia in 2012. Since then, over 1000 cell lines have been collected and screened with integrated information for drug sensitivity, mRNA expression and genomic variation. Several other institutes and pharmaceutical companies have also established high throughput cell line screening programs. These programs have accelerated the drug development process.

Cell line screening is not only helpful in determining drug sensitivity, but also helpful in identifying biomarkers and mechanisms of action associated with candidate drugs. However, the screening process is extremely laborious. Traditional methods screen each drug in each cell line. Thus, screening multiple drugs in multiple cell lines is tedious and costly work, even with the help of a robotic liquid handling system. For example, if for each compound, five doses plus a vehicle control are tested in triplicate, then each cell line needs to be grown in 18 plates or wells. After treatment, cells in each plate or well need to be analyzed separately. Thus, if testing occurs in 60 cell lines, over 1000 samples would need to be analyzed for each drug. As this example makes clear, it becomes virtually impossible to perform routine screening with larger numbers of drug candidates and larger numbers of cell lines due to the massive number of individual samples that would be required.

Recently, the Broad Institute developed a method to screen multiple cell lines in a mixture, which is known as PRISM (profiling relative inhibition simultaneously in mixtures). In this method, each cell line is labeled with an exogenous tag, which is a specific, unique DNA sequence referred to as a “barcode.” The proportion of each “barcoded” cell line in the mixture can be determined by genomic analysis. This method is highly efficient and has been successfully used in a number of studies.

As an inherent limitation, the cell lines used for PRISM require barcoding before they can be used, which is tedious and time-consuming. Additionally, the process of barcode insertion and stable-cell line selection could change the properties of these engineered cell lines. This leads to a concern about whether screening results obtained from the engineered cell lines are truly representative of drug responses that would occur in parent, unmodified, cell lines.

Accordingly, there remains a need in the art for tools that allow for the benefits of multiple mixed cell line screening, without the limitations associated with use of engineered cell lines, such as those requiring introduction of a “barcode.”

### SUMMARY

The presently disclosed subject matter meets some or all of the above-identified needs, as will become evident to those of ordinary skill in the art after a study of information provided in this document.

This summary describes several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter includes methods for screening a test compound against multiple cell lines. In particular, certain embodiments of the presently disclosed subject matter include methods for cell line screening that make use of endogenous single nucleotide polymorphisms (SNPs) to assess the efficacy of a test compound against multiple cell lines. In some embodiments, a method for screening a test compound against multiple cell lines comprises: treating a mixture including cells from multiple cell lines with the test compound; identifying allele frequencies of SNPs in the treated mixture and a control mixture including cells from the same cell lines as present in the treated mixture; estimating a proportion of each individual cell line in both the treated mixture and the control mixture; and quantifying the effect of the test compound on each individual cell line using the estimated proportion of each cell line in the treated mixture and the estimated proportion of each cell line in the control mixture. To eliminate the expense, time investment, and potential for cell property alteration associated with barcode labeling, in some embodiments, the cells from the multiple cell lines in the treated mixture and the control mixture are free of exogenous barcode nucleotides.

Estimating the proportion of each individual cell line in the treated mixture and the control mixture can, in some embodiments, include performing deconvolution of the treated mixture and the control mixture. In some embodiments, deconvolution of the treated mixture and the control mixture is performed by one or more processors. In some embodiments deconvolution of the treated mixture and the control mixture is based, at least in part, on: (i) a comparison of the identified allele frequencies of SNPs in the treated mixture and in the control mixture to a predetermined number of SNPs; and (ii) one or more unique patterns associated with each respective cell line. In some embodiments, the predetermined number of SNPs to which the identified allele frequencies of SNPs in the treated mixture and the control mixture are compared is equal to a total number of SNPs identified across the multiple cell lines. In other embodiments, the predetermined number of SNPs is equal to a subset of the total number of SNPs identified across the multiple cell lines. In some embodiments, quantifying the effect of the test compound on each respective cell line includes generating a value for each cell line of the multiple cell lines which is indicative of the inhibitory effect of the test compound on the cell line.

Further provided, in some embodiments of the presently disclosed subject matter, are systems for screening a test compound against multiple cell lines. In some embodiments, such systems include one or more processors and memory storing instructions that, when executed by the one or more processors, cause the one or more processors to perform operations that include: comparing data corresponding to allele frequencies of SNPs in a first mixture treated with a test compound and including cells from multiple cell lines to a predetermined number of SNPs; comparing data corresponding to allele frequencies of SNPs in a second mixture with cells from the multiple cell lines to the predetermined number of SNPs; estimating a proportion of each of the multiple cell lines in the first mixture by performing deconvolution of the first mixture; estimating a proportion of each of the multiple cell lines in the second mixture by performing deconvolution of the first mixture; and quantifying the effect of the test compound on each of the multiple cell lines based on the estimated proportion of each of the cell lines in the first mixture and the second mixture.

Systems and methods for identifying a proportion of a particular cell line in a mixture including multiple cell lines are also provided.

In some embodiments of the systems and methods disclosed herein, each cell line of the multiple cell lines is a cancer cell.

Further features and advantages of the presently disclosed subject matter will become evident to those of ordinary skill in the art after a study of the description, figures, and non-limiting examples in this document.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 is a flow diagram showing an overview of an exemplary single nucleotide polymorphism (SNP)-based mixed cell screen (SMICS) platform in accordance with the present disclosure. Multiple cell lines, each containing a unique pattern of single nucleotide polymorphisms (SNPs) (identified as “A”, “B”, and “C” in FIG. 1), are mixed in a pool and treated with either drug or control vehicle. Sequencing is performed to identify allele frequencies of SNPs in the mixture samples. Such data along with unique SNP patterns of individual cell lines are then used to deconvolute the fraction of each cell line in the mixture samples. By comparing the cell count between drug-treated and control samples, the drug-sensitive/resistant status of each cell line is concluded.

FIG. 2 shows the chemical structure of testing compound 4-((2,6-difluorophenyl)ethynyl)-N,N-dimethylaniline (DEDA) used in certain studies of the present disclosure.

FIG. 3 provides the results of a Western blot showing that DEDA (1 μM) inhibited c-Myc expression in C3A but not in SK-Hep1 liver cancer cells.

FIGS. 4A-B show a table and images illustrating the effects of DEDA on different liver cancer cells. (A) Table showing the effects of DEDA (1 μM) on cell proliferation of different liver cancer cell lines. (B) Images of DEDA effects on each liver cancer cell lines and on a mixture of six cell lines.

FIGS. 5A-C show a table and graphs illustrating DEDA inhibition effect quantification across six cell lines based on the SMICS platform. (A) Table showing estimated cell line proportions in mixture samples from DEDA and dimethylsulfoxide (DMSO) groups. The 95% confidence interval is presented in parenthesis. (B) Graph showing the comparison of the ratio of cell counts (DEDA vs. DMSO) of six individual cell lines between cell proliferation assay and whole exome sequencing (WES) in a mixture including multiple cell lines. Results averaged across 3 replicates. (C) Graph showing the comparison of the standard error of the estimation using mixtures and individual cell proliferation assays (SMICS=left bar; cell proliferation assay=right bar for each cell line).

FIGS. 6A-B show a table and graphs illustrating the validation of a cell line mixture deconvolution (CLMD) algorithm of the SMICS platform based on simulation studies, where next-generation sequencing (NGS) data for samples of mixtures of six cell lines with DEDA or DMSO treatment were simulated by using parameters specified according to experimental data. (A) Table showing the performance of the CLMD algorithm for estimating the cell line proportion and drug inhibition effect. Bias is the difference between the estimated value and true value used to simulate the data; cp is the coverage probability of 95% confidence interval; rmse is the root-mean-square error. (B) Graphs showing the performance of the CLMD algorithm when a subset of SNPs is used. All results were averaged over 1000 simulations.

FIG. 7 is a graph showing scalability evaluation of the SMICs platform based on simulations. NGS data for samples of mixtures of NCI60 cell lines with aurone-5a or DMSO treatment were simulated based on experimental data of aurone-5a inhibition effect on those cell lines. The CLMD algorithm was applied to estimate the drug inhibition effect (in terms of ratio of cell counts between aurone-5a and DMSO groups) for each cell line. The estimated value was compared to the true value used to simulate the data. The error bar indicates the standard error of the estimation.

FIG. 8 is a graph showing the comparison of the ratio of cell counts (DEDA vs. DMSO) of six individual cell lines between a panel of 360 selected SNPs and 71,488 SNPs identified via WES in a mixture including multiple cell lines.

FIG. 9 is a flow diagram showing an exemplary method of screening a test compound against multiple cell lines.

FIG. 10 is a schematic diagram showing an example computing device which may be utilized in methods and systems disclosed herein.

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11 (9):1726-1732).

Although any methods, devices, and materials similar or equivalent to those described herein can be used in the practice or testing of the presently-disclosed subject matter, representative methods, devices, and materials are described herein.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As will be recognized by one of ordinary skill in the art, the terms “suppression,” “suppressing,” “suppressor,” “inhibition,” “inhibiting” “inhibitor,” or “inhibitory effect” do not necessarily refer to a complete elimination of a value in all cases. Rather, the skilled artisan will understand that the term “suppressing” or “inhibiting” refers to a reduction or decrease in a measured value, qualitatively or quantitatively. Such reduction or decrease can be determined relative to a control or a prior status of a subject. In some embodiments, the reduction or decrease relative to a control or the prior status of a subject can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease.

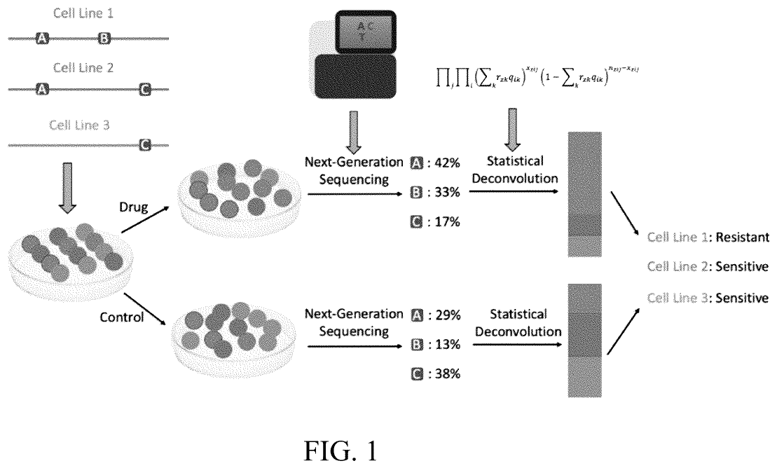

The presently disclosed subject matter is based, at least in part, on the development of an exemplary single nucleotide polymorphism (SNP)-based mixed cell screen (SMICS) platform (FIG. 1) which makes use of the unique SNP patterns associated with, and endogenous to the cells of, respective cell lines and deconvolution to estimate the proportion of individual cell lines present within a mixture including multiple cell lines. By leveraging endogenous SNPs as the means of identifying and differentiating the respective cell lines in the mixture, the SMICS platform eliminates the need to label the respective cell lines with exogenous nucleotide tags (or nucleotide barcodes), such as oligonucleotide tags, and thus the costs, time investment, and potential for altering the properties of the cells of the respective cell lines associated with such labeling. During the course of development of the SMICS platform, it was surprisingly discovered that the efficacy of a test compound (e.g., a candidate therapeutic drug) against the respective cell lines present within a mixture can be accurately quantified utilizing the estimated proportion of each respective cell line in the treated mixture and the estimated proportion of each respective cell line in a control mixture including corresponding cell lines. Moreover, it was surprisingly discovered that accurate estimates of respective cell lines in a mixture and accurate quantification of test compound efficacy can still be achieved even when utilizing SNP panels comprising a small subset of SNPs (i.e., small relative to the total number of identified SNPs across each of the multiple cell lines within a mixture) as a basis of comparison for identified allele frequencies of SNPs in the treated mixture and the control mixture, as further discussed below.

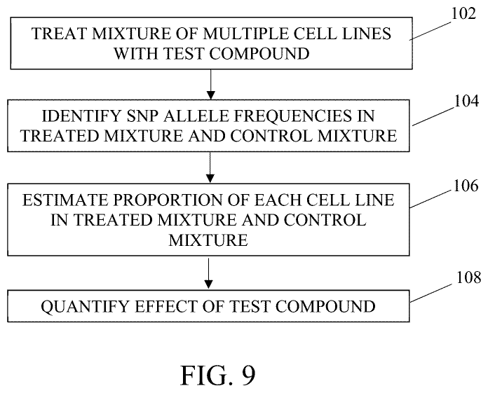

Accordingly, in one aspect, the presently disclosed subject matter includes a method of screening a test compound against multiple cell lines. In one embodiment, the method commences by treating a mixture including cells from multiple cell lines with a test compound, as indicated by block 102 in FIG. 9. Each respective cell line has one or more unique patterns of SNPs associated therewith relative to the other cell lines present in the mixture. To eliminate the expense, time investment, and potential for cell property alteration associated with barcode labeling, in some embodiments, the cells from each respective cell line in the treated mixture are free of exogenous barcode nucleotides.

Following treatment with the test compound, the allele frequencies of SNPs in both the treated mixture and a control mixture including cells from the same cell lines as present in the treated mixture are identified, as indicated in block 104 in FIG. 9. In some embodiments, the control mixture includes cells which are treated with a control compound. In some embodiments, the control compound is dimethylsulfoxide (DMSO). DMSO can serve as a solution (vehicle) for other compounds. In some embodiments, identification of allele frequency of SNPs in the treated sample and the control sample is performed, at least in part, utilizing next generation sequencing (NGS). In some embodiments, identification of the allele frequencies of SNPs in the treated sample and the control sample is performed, at least, in part, utilizing whole exome sequencing (WES) and/or whole genome sequencing (WGS) by short-or long-read sequencing technologies.

The proportion of each cell line present in both the treated mixture and the control mixture is then estimated by performing deconvolution of the treated mixture and the control mixture, as indicated by block 106 in FIG. 9. In some embodiments, the estimated proportion of each cell line present in the treated mixture and the estimated proportion of each cell line present in the control mixture may be provided as a numeric value. Accordingly, in some embodiments, estimating the proportion of each cell line present in n both the treated mixture and the control mixture may include generating a numeric value indicative of the proportion of each cell line present in the treated mixture and generating a numeric value indicative of the proportion of each cell line in the control mixture. In some embodiments, deconvolution of the treated mixture and the control mixture is based, at least in part, on: (i) a comparison of the identified allele frequencies of SNPs in both the treated mixture and in the control mixture to a predetermined number of SNPs (i.e., the identified allele frequencies of SNPs of the treated mixture and the control mixture are independently compared to a predetermined number of SNPs); and (ii) the one or more patterns of SNPs associated with each respective cell line in the mixture. In some embodiments, during deconvolution, a library of the multiple cell lines may be utilized to facilitate mapping of the allele frequencies of SNPs identified in the treated mixture and the control mixture to individual cell lines. In this regard, the library of multiple cells will typically identify the one or more unique patterns of SNPs associated with each particular cell line with the particular cell line to which it corresponds. The predetermined number of SNPs to which the identified allele frequencies of SNPs in the treated mixture and the control mixture are compared will typically include SNPs that correspond to at least one pattern of SNPs unique to each respective cell line in the mixture. That is, the predetermined number of SNPs serving as the basis of comparison will encompass a sufficient number and selection of SNPs as to permit identification of each respective cell line in the treated mixture and in the control mixture based on the observance of one or more patterns of SNPs unique to the respective cell line in the identified allele frequencies.

In some embodiments, the predetermined number of SNPs to which the identified allele frequencies of SNPs in the treated mixture and the control mixture are compared during deconvolution is equal to a total number of SNPs identified across the multiple cell lines. In some embodiments, the total number of SNPs may be determined during WES of the multiple cell lines. However, as noted above and further evidenced in the discussion below, during the course of development of the SMICS platform, the inventors surprisingly discovered that accurate cell line proportion estimates and test compound efficacy quantification can still be realized utilizing significantly fewer SNPs than the total number of SNPs identified across the multiple cell lines. Accordingly, in some embodiments, the predetermined number of SNPs to which the identified frequencies of SNPs in the treated mixture and the control mixture are compared to is equal to a subset of the total number of SNPs identified across the multiple cell lines. In some embodiments, the subset of the total number of SNPs may range from about 50 to about 800 SNPs. In some embodiments, the subset of the total number of SNPs is 400 or fewer SNPs. In some embodiments, the subset of the total number of SNPs may range from about 360 to 800 SNPs. In some embodiments, deconvolution of the treated mixture and the control mixture is carried out utilizing a cell line mixture deconvolution (CLMD) algorithm consistent with that described below.

Following estimation of the proportion of each respective cell line in the treated mixture and the proportion of each respective cell line in the control mixture, the exemplary method for screening the test compound against multiple cell lines concludes, in this embodiment shown in FIG. 9, by quantifying the effect of the test compound on each respective cell line is quantified, as indicated by block 108 in FIG. 9. In this regard, a numeric value is generated for each respective cell line, where the numeric value is indicative of the effect of the test compound on that respective cell line. In some embodiments, the numeric value for a particular cell line is generated based, at least in part, on the ratio of estimated proportion of the cell line in the treated mixture to the estimated proportion of the cell in the control mixture. In some embodiments, the numeric value for a particular cell line is indicative of the inhibitory effect of the test compound on that particular cell line.

In some embodiments, estimating the proportion of each respective cell line in the treated mixture and the control mixture, and/or quantifying the effect of the test compound on each respective cell line may be performed by one or more processors of a computing device.

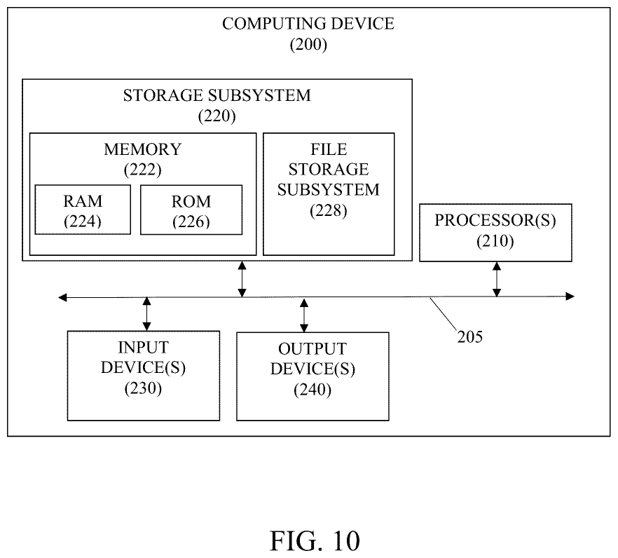

FIG. 10 is an exemplary computing device 200 which may be utilized to estimate the proportion of each respective cell line in the treated mixture and the control mixture and/or to quantify the effect of the test compound on each respective cell line. As shown, the computing device 200 includes one or more processors 210 configured to execute one or more programs corresponding to instructions stored in memory 222 to perform the operations of the one or more processors 210 disclosed herein. In this regard, the one or more processors 210 is operably connected to the memory 222 via a bus 205, such that instructions stored in memory 222 can be communicated to, and subsequently executed by, the one or more processors 210. As shown, memory 222 can include random access memory (RAM) 224, which provides storage for instructions and data during while the one or more programs are being executed by the one or more processors 210. Memory 222 can also include read only memory (ROM) 228 to provide non-volatile storage. In some embodiments, the memory 222 may be a component of a larger storage subsystem 220 operably connected to the one or more processors 210 via the bus 205. In addition to memory 222, the storage subsystem 220 may also include a file storage subsystem 228 that facilitates persistent storage for program and data files and can include data storage devices, such as solid state drives (SSD) or hard disk drives (HDD). As shown, in some embodiments, the one or more processors 210 can also be operably connected to one or more input devices 230 (e.g., keyboard, mouse, CD-ROM drive, etc.) for inputting information into the computing device 200 and one or more output devices 240 for outputting information from the computing device 200 (e.g., display, printer, etc.). Although certain examples of input devices are provided herein, to alleviate any doubt, it should be appreciated that the term “input device” as used herein is intended to encompass any suitable means and devices for inputting information into the computing device 200. Similarly, it should be appreciated that, while certain examples of output devices may be provided herein, the term “output device” as used herein is intended to encompass any suitable means or devices to output information from the computing device 200 to a user or another computing device.

It is appreciated that each method step described herein can also be characterized as an operation performed by the one or more processors 210 of the computing device 200, unless specified otherwise or context precludes. Accordingly, and referring now to FIGS. 9 and 10, in embodiments in which the estimation of the proportion of each respective cell line in the treated mixture and the control mixture is performed via one or more processors 210, the memory 222 will include a program corresponding to instructions, which, when executed by the one or more processors 210, cause the one or more processors 210 to perform deconvolution of the treated mixture and the control mixture. In this regard, instructions for carrying out the CLMD algorithm disclosed herein may be stored in the memory 222 and subsequently executed by the one or more processors 210. The memory 222 may thus include instructions, which, when executed by the one or more processors, cause the one or more processors 210 to perform operations that include: comparing data corresponding to allele frequencies of SNPs identified in the treated mixture to a predetermined number of SNPs to identify which of the predetermined number of SNPs are present in the treated mixture; comparing data corresponding to allele frequencies of SNPs identified in the control mixture to the predetermined number of SNPs to identify which of the predetermined number of SNPs are present in the control mixture; estimating a proportion of each respective cell line in the treated mixture; and estimating a proportion of each respective cell line in the control mixture.

Embodiments in which the data corresponding to the allele frequencies of SNPs in the treated mixture and/or the data corresponding to the allele frequencies of SNPs in the control mixture are generated, in whole or in part, locally on the computing device 200, as well as embodiments in which such data is generated externally, in whole or in part, and subsequently supplied to the computing device 200 (e.g., via the one or more input device(s) 230 and/or through a network connection placing the computing device 200 in communication with another device) for subsequent processing, are contemplated herein. For instance, in some embodiments, sequence data for the treated mixture and the control mixture may be initially generated by a sequencing device (e.g., a whole exome sequencer), transmitted to the computing device 200, and then processed by the one or more processors 210 to identify the allele frequencies of SNPs in the treated mixture and the control mixture. In this regard, embodiments in which the one or more processors 210 execute instructions stored in memory 222 that cause the one or more processors 210 to perform the operations of identifying allele frequencies of SNPs in the treated mixture and identifying the allele frequencies of SNPs in the control mixture are contemplated herein. In some embodiments, such program(s) may facilitate manipulation of sequence data received by the computing device 200 prior to identification of the allele frequencies present in the treated mixture and control mixture when executed by the one or more processors 210.

To estimate the proportion of each respective cell line in the treated mixture, the one or more processors 210 may utilize the results derived from the comparison of the data corresponding to the allele frequencies of SNPs in the treated mixture to the predetermined number of SNPs and one or more unique patterns of SNPs associated each respective cell line. In some embodiments, a library of the multiple cell lines, which identifies the one or more unique patterns of SNPs associated with each particular cell line with the particular cell line to which it corresponds, may be stored on the computing device 200 and accessible to the one or more processors 210. In such cases, the one or more processors 210 may reference the results of the comparison of data corresponding to the allele frequencies of SNPs in the treated mixture to the predetermined number of SNPs against the library to estimate the proportion of each respective cell line present in the treated mixture. The proportion of each respective cell line in the control mixture may be similarly estimated by the one or more processors 210. In some embodiments, the estimate of the proportion of each cell line in the treated mixture and the control mixture may be embodied as a numeric value generated by the one or more processors 210 which is indicative of the proportion of the cell line in the treated mixture and control mixture, respectively.

In some embodiments, in which the effect of the test compound on each respective cell line is quantified via the one or more processors 210, the memory 222 will include instructions, which, when executed by the one or more processors 210, cause the one or more processors 210 to generate a numeric value for each cell line that is indicative of the effect of the test compound on the cell line. In some embodiments, the one or more processors 210 may generate the numeric value for each cell line based, at least in part, on the ratio of the numeric value indicative of the estimated proportion of the cell line in the treated mixture to the numeric value indicative of the estimated proportion of the cell line in the control mixture. In some embodiments, the numeric value indicative of the effect of the test compound on a particular cell line generated by the one or more processors 210 may correspond to the inhibitory effect of the test compound on that particular cell line.

As evidenced by the discussion above, the presently disclosed subject matter also includes a system for screening a test compound against multiple cell lines, which comprises the one or more processors 210; and memory 222, which stores instructions that, when executed by the one or more processors, cause the one or more processors 210 to perform some or all of the operations described above for the one or more processors 210.

It should be appreciated that some or all of the techniques disclosed herein may find utility outside the screening of a test compound against multiple cell lines. For instance, as reflected in the discussion above, techniques disclosed herein can be utilized to determine the proportion of a particular cell line within a mixture of multiple cell lines. Accordingly, in another aspect, the presently disclosed subject matter also includes a method for identifying a proportion of a particular cell line in a mixture including multiple cell lines. In some embodiments, the proportion of the particular cell line within the mixture of multiple cell lines is identified by carrying out some or all of the techniques and steps described above for steps 102, 104, and/or 106 of FIG. 9 in connection with the treated mixture or the control mixture for the method of screening a test compound against multiple cell lines. For instance, in some embodiments, the method for identifying a proportion of a particular cell line in a mixture includes: identifying allele frequencies of SNPs in a mixture including multiple cell lines; and estimating the proportion of the particular cell line in the mixture by performing deconvolution of the mixture based, at least in part, on: (i) a comparison of the identified allele frequencies of SNPs in the mixture to a predetermined number of SNPs; and (ii) one or more unique patterns of SNPs associated with each of the multiple cell lines present in the mixture.

As noted, each method step described herein can also be characterized as an operation performed by the one or more processors 210 of the computing device 200, unless specified otherwise or context precludes. Accordingly, in yet another aspect, the presently disclosed subject matter also includes a system for identifying a proportion of a particular cell line in a mixture, which comprises the one or more processors 210 and memory 222, which includes instructions that, when executed by the one or more processors 210 cause the one or more processors 210 to perform operations corresponding to the above-noted method steps for the method for identifying a proportion of a particular cell line in a mixture including multiple cell lines.

It is appreciated that each operation performed by the one or more processors 210 described herein can also be characterized as a method step, unless otherwise specified or context precludes.

The present disclosure also contemplates the use of one or more non-transitory computer readable storage media storing computer instructions executable by one or more processors to perform some or all of the operations described herein for the one or more processors 210.

In some embodiments of the methods and systems disclosed herein, each cell line of the multiple cell lines is a cancer cell line. In some embodiments of the methods and systems disclosed herein, each cell line of the multiple cell lines is a liver cancer cell line. In some embodiments of the methods and systems disclosed herein, the multiple cell lines comprise one or more cell lines selected from the group consisting of: Huh7, C3A, PLC/PRF/5, SNU449, and Sk-Hep1. In some embodiments of the methods and systems disclosed herein, the multiple cell lines comprise at least six cell lines. In some embodiments of the methods and systems disclosed herein, the cells corresponding to the multiple cell lines may be acquired from a subject in vivo using known cell collection processes.

With respect to the presently-disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary applications for the disclosed methods and systems are contemplated herein.

The presently disclosed subject matter is further illustrated by the following specific but non-limiting examples. The following examples may include compilations of data that are representative of data gathered at various times during the course of development and experimentation related to the present invention.

### EXAMPLES

### Materials and Methods

The liver cancer cell lines, Huh7, C3A, PLC/PRF/5, SNU449, SNU475 and SK-Hep1, were purchased from the American Type Culture Collection (ATCC). The cells were cultured in DMEM (ATCC®30-2002) containing 10% (v/v) Fetal Bovine Serum (FBS) (Sigma F0926) and grown in an incubator at 37° C. with 5% CO₂. The halogenated diarylacetylene, 4-((2,6-difluorophenyl) ethynyl)-N,N-dimethylaniline (DEDA), was synthesized and characterized as previously described.^{18,19}

### Cell Culture

For proliferation assays of single cell line, cells were seeded in 12-well plates (2×10⁵ cells per well) and incubated overnight. The following day, DEDA (1 μM solution in DMSO) was added to each well with DMSO alone as a control. Each experiment was performed in quadruplicate. After 30 h, cells were analyzed under a reverse microscope. Cell viability and number were analyzed using the Vi-Cell XR Cell Viability Analyzer (Beckman Coulter).

### Western Blot

Cells were grown in six-well plates and lysed in 0.5 mL/well lysis buffer (50 mM HEPES, 100 mM NaCl, 2 mM EDTA, 1% glycerol, 50 mM NaF, 1 mM Na₃VO₄, 1% Triton X-100, with protease inhibitors). Cell lysates were separated by a 10% SDS-PAGE gel and transferred to an Immobilon PVDF membrane. The protein levels of c-Myc and GAPDH (loading control) were analyzed using antibodies that recognize c-Myc (Epitomics, #1472-1) and GAPDH (GeneTex, #627408).

### Whole Exome Sequencing

A mixture of the six liver cancer cell lines was cultured in six-well plates (6×10⁵ cells per well). Three wells were treated with 3 μL of DMSO and the other three wells were treated with 3 μL of DEDA (1 μM). After 30 h, the cells were collected, and genomic DNA was isolated using the QIAamp DNA Mini Kit from QIAGEN. The whole exome sequencing (100×) was performed at Novogen.

### SNP Calling

Sequencing reads were trimmed and filtered using Trimmomatic²⁴ (v0.39) aligned to human reference genome b37/hg19 using BWA (v0.7.17). PCR duplicates were removed using Picard²⁵ (v2.20.0). The Genome Analysis Toolkit (GATK²⁶ v4.1.2.0) was used for base quality score recalibration. For each individual liver cancer cell line, HaplotypeCaller was run in gVCF output mode, producing an intermediate single sample gVCF for joint genotyping. Individual gVCFs were combined and the final joint genotyping step was performed on all six cell lines. GATK variant quality score recalibration was run for SNP and INDEL separately with default filters as suggested by the GATK documentation. To generate the reference set from the six cell lines, we excluded INDELs from all future analysis and kept only SNPs with at least 30× coverage and were called in at least one cell line. Germline mutation calling for the cell line mixture samples was performed using bcftools²⁷ (v1.9) mpileup and were limited to the SNP loci identified in the reference set. SNP loci with the same genotype across all six cell lines or <=30× coverage in any of the 6 samples were further removed. The final matrix for cell line mixture deconvolution was constructed including the germline mutation loci information and allelic depth for each individual sample.

### Cell Line Mixture Deconvolution (CLMD) Algorithm

The CLMD algorithm considers a probabilistic model on the allele frequencies of SNPs to estimate the proportion of each cell line in mixture samples, and then to infer drug inhibition effect on a cell line. Coding for the SMICS implementing is available at https://github.com/Markey/BBSRF/SMICS, which is incorporated herein by reference in its entirety. Let n_{zij} be the total number of reads and X_{zij} be the number of reads supporting the minor allele at SNP site i in the jth mixture replicate of the zth experimental group (z=1 for the drug-treated group or 0 for the DMSO group). We have X_{zij}˜Bin(n_{zij},p_{zi}), where p_{zi}=Σₖ₌₁^{K}r_{zk}qᵢₖ, r_{zk} is the proportion of the kth cell line in the zth experimental group, and qᵢₖ is the minor allele frequency of SNP i in the kth cell line for k=1, . . . ,K. The qᵢₖ takes value 0, 0.5 or 1 depending on whether the genotype at site i is reference, heterozygous mutation, or homozygous mutation. We use the following ratio of cell counts (e.g., DEDA to DMSO) to quantify the drug efficacy on the kth cell line:

$${RCC}_{k} = {r}_{1 k} {c}_{1} / {r}_{0 k} {c}_{0} ,$$

where c₀ is the averaged total cell count in DMSO samples and c₁ is the averaged total cell count in drug-treated samples. To estimate r_{zk} based on n_{zij} and X_{zij} from WES of cell line mixtures, consider the following log-likelihood function

$$l ( {r}_{z 1} , ⋯ , {r}_{zK} ) = {\sum }_{j} {\sum }_{i} \{ {x}_{zij} log ( {p}_{zi} ) + ( {n}_{zij} - {x}_{zij} ) log ( 1 - {p}_{zi} ) \} ,$$

where x_{zij} is the observed value of X_{zij}. The maximum likelihood estimate of r_{zk} can be obtained by maximizing l(r_{z1}, . . . ,r_{zk}) under the constraints that 0<r_{zk}<1 and Σₖ₌₁^{K}r_{zk}=1. To simplify the calculation, we consider the following reparameterization to convert the constrained maximization problem into an unconstrained maximization problem:

$${r}_{zk} = \frac{exp ( {ω}_{zk} )}{{\sum }_{m = 1}^{K - 1} exp ( {ω}_{zm} ) + 1} ,$$

$$k = 1 , ⋯ , K - 1 ,$$

$$and$$

$${r}_{zK} = \frac{1}{{\sum }_{m = 1}^{K - 1} exp ( {ω}_{zm} ) + 1} .$$

The log-likelihood can be rewritten as a function of ω's, referred to as l(ω_{z1}, . . . ,ω_{zK-1}). The estimate of ω_{zk}, \hat{ω}_{zk}, is obtained by maximizing l(ω_{z1}, . . . ,ω_{zK-1}) using the “trust” package in R. Then \hat{ω}_{zk} is transformed back to get the estimate of r_{zk}, \hat{r}_{zk}, and estimate of RVCₖ, [symbol] .

The variance estimates of \hat{r}_{zk} and [symbol] are obtained based on the delta method. Let Ĥ_{z} be an estimated Hessian matrix of l(ω_{z1}, . . . ,ω_{zK-1}), where the (k, m) element of Ĥ_{z} is

$${\sum }_{j} {\sum }_{i} \{ - \frac{{x}_{zij}}{{\hat{p}}_{zi}^{2}} - \frac{{n}_{zij} - {x}_{zij}}{{( 1 - {\hat{p}}_{zi} )}^{2}} \} {\{ {\sum }_{v} ( {q}_{ik} - {q}_{iv} ) {\hat{r}}_{zk} {\hat{r}}_{zv} \}}^{2} + \{ \frac{{x}_{ij}}{{\hat{p}}_{\dot{z} i}} - \frac{{n}_{ij} - {x}_{ij}}{1 - {\hat{p}}_{\dot{z} i}} \} \{ {\sum }_{v} ( {q}_{ik} - {q}_{iv} ) {\hat{r}}_{zk} {\hat{r}}_{zv} ( 1 - 2 {\hat{r}}_{zk} ) \}$$

$$for$$

$$k = m$$

$$or$$

$${\sum }_{j} {\sum }_{i} \{ - \frac{{x}_{zij}}{{\hat{p}}_{zi}^{2}} - \frac{{n}_{zij} - {x}_{zij}}{{( 1 - {\hat{p}}_{zi} )}^{2}} \} \{ {\sum }_{v} ( {q}_{ik} - {q}_{iv} ) {\hat{r}}_{zk} {\hat{r}}_{zv} \} \{ {\sum }_{v} ( {q}_{im} - {q}_{iv} ) {\hat{r}}_{zm} {\hat{r}}_{zv} \} + \{ \frac{{x}_{ij}}{{\hat{p}}_{\dot{z} i}} - \frac{{n}_{ij} - {x}_{ij}}{1 - {\hat{p}}_{\dot{z} i}} \} \{ {\sum }_{v} ( 2 {q}_{iv} - {q}_{ik} - {q}_{im} ) {\hat{r}}_{zk} {\hat{r}}_{zm} {\hat{r}}_{zv} \}$$

for k≠m with \hat{p}_{zi}=Σₖ₌₁^{K}\hat{r}_{zk}qᵢₖ. Based on the asymptotic properties of maximum likelihood estimator, the estimated variance of \hat{ω}_{z}=(\hat{ω}_{z1}, . . . ,\hat{ω}_{zK-1})^{T} is (−Ĥ_{z})⁻¹. Thus, based on the delta method, the estimated variance of \hat{r}_{z}=(\hat{r}_{z1}, . . . ,\hat{r}_{zK})^{T} is \hat{B}_{z}(−Ĥ_{z})⁻¹\hat{B}_{z}^{T}, where \hat{B}_{z} is an estimated K×(K−1) Jacobian matrix of r_{z} with the (k, m) element equal to \hat{r}_{zk}(1−\hat{r}_{zk}) for k=m or −ê_{zk}\hat{r}_{zm} for k≠m. Likewise, the estimated variance of [symbol] =( [symbol] , . . . , [symbol] )^{T} is (c₁²/c₀²)\hat{D}(−Ĥ)⁻¹\hat{D}^{T}, where Ĥ=Diag(Ĥ₀,Ĥ₁), \hat{D}=(\hat{D}₀,\hat{D}₁) is an estimated K×(2K−2) Jacobian matrix of IR with the (k, m) element of \hat{D}₀ equal to \hat{r}₀ₖ(1−\hat{r}₀ₖ)/\hat{r}₁ₖ for k=m or −\hat{r}₀ₖ\hat{r}₀ₘ/\hat{r}₁ₖ for k≠m and the (k, m) element of \hat{D}₁ equal to \hat{r}₀ₖ(1−1/\hat{r}₁ₖ) for k=m or \hat{r}₀ₖ\hat{r}₁ₘ/\hat{r}₁ₖ for k≠m.

### Simulating NGS Data of Six Liver Cancer Cell Lines

NGS data was simulated mimicking the real WES data of the mixture of the six cell lines. Each simulated dataset contained three samples from each of DEDA-treated and DMSO-treated control groups, where each sample is a mixture of six cell lines. We simulated data of the 71,488 SNPs that were identified from WES of the six cell lines. The genotypes of cell lines and the sequencing depth at each SNP site, as well as the proportion of each cell line in DEDA-treated and control samples were specified according to the real WES data. Specifically, for SNP i (i=1, . . . , 71,488) in sample j of group z, the total number of reads, n_{zij}, was specified based on the real WES data, and the number of reads containing the alternative allele, X_{zij}, was simulated based on a binomial distribution Bin(n_{zij},p_{zi}) with parameter p_{zi}=Σₖ₌₁⁶r_{zk}qᵢₖ, where qᵢₖ was specified according to the observed genotype in the real data of the kth cell line and r_{zk} was specified according to the estimated proportion of the kth cell line in the real cell line mixture WES data.

### Simulating NGS Data of NCI60 Cell Lines

We focused on the 500,000 SNPs that were included in the Affymetrix GeneChip® Human Mapping 500K Array, where the genotype of each cell line at each SNP site was specified based on the array data of NCI60 cell lines downloaded from the CellMiner website²⁸. We simulated NGS data of a mixture of the NCI60 cell lines for six samples (three Aurone-5a-treated samples and three DMSO samples). The proportion of each line in a Aurone-5a-treated sample was specified according to the drug inhibition data of that cell line from screen of Aurone-5a through NCI60 program.²¹ The proportion of each cell line in the mixture was equally set to 1/60 in a DMSO-treated sample. Similar to the last subsection, binomial distributions were used to simulate data at those SNP sites. The sequencing depth at each SNP site was set to 100×.

### Simulating SNP-Panel Approach

Using the results obtained from Whole-exome sequencing (WES) approach as a gold standard, an SNP-panel approach was tested using a simulation method. In the WES approach, a total of 71,488 SNPs were used for the estimation of drug inhibition effect in SMICS platform. In the SNP-panel approach, a subset of the 71,488 SNPs is selected and utilized to estimate the proportion of individual cell lines within a mixture of cell lines and quantify drug inhibition.

### Results

### SMICS Platform

Unlike traditional platforms where each well on multi-well plates contains only one cell line, the SMICS platform (FIG. 1) co-cultures multiple cell lines in each well. NGS determines the allele frequencies of SNPs in the mixture samples. Based on the unique pattern of germline SNPs of each individual cell line, SMICS uses a cell line mixture deconvolution (CLMD) statistical algorithm to estimate the proportion of each cell line in the mixture samples. CLMD considers a probabilistic model on the allele frequencies of SNPs, where the observed number of reads containing the minor allele at each SNP site follows a binomial distribution with a parameter depending on the proportions of the cell lines and their associated minor allele frequencies. Based on the probabilistic model and using the maximum likelihood method, CLMD estimates the underlying proportion of each cell line in the mixture samples. A detailed description of CLMD is provided above. By calculating the ratio of cell counts between drug-treated and DMSO samples, i.e., estimated number of cell counts from a cell line in drug-treated samples (the proportional amount times the total number of cells) divided by that in DMSO samples, SMICS determines the level of drug inhibition for each cell line in the mixtures.

### Platform Validation Based on Liver Cancer Cell Lines

Liver cancer is a leading cause of cancer deaths worldwide, accounting for >700,000 deaths each year. It is particularly unfortunate that liver cancer in the United States remains a neglected cancer in comparison with other types of cancer, perhaps best exemplified by the absence of any liver cancer cell line in the NCI60 screening program. The drugs currently approved by the FDA for liver cancer, sorafenib and regorafenib, only extend the median survival time for patients with liver cancers by a few months.

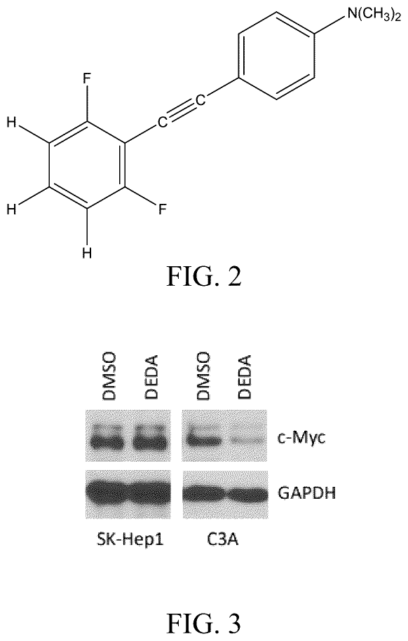

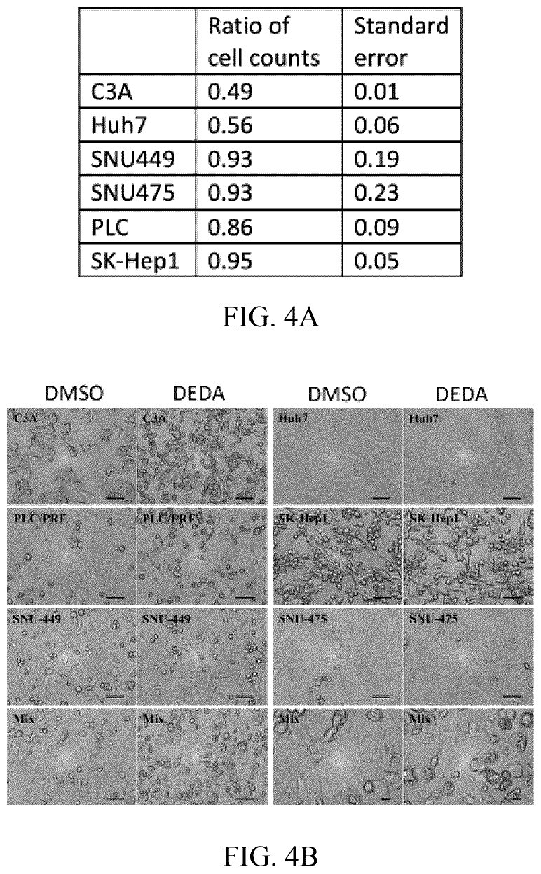

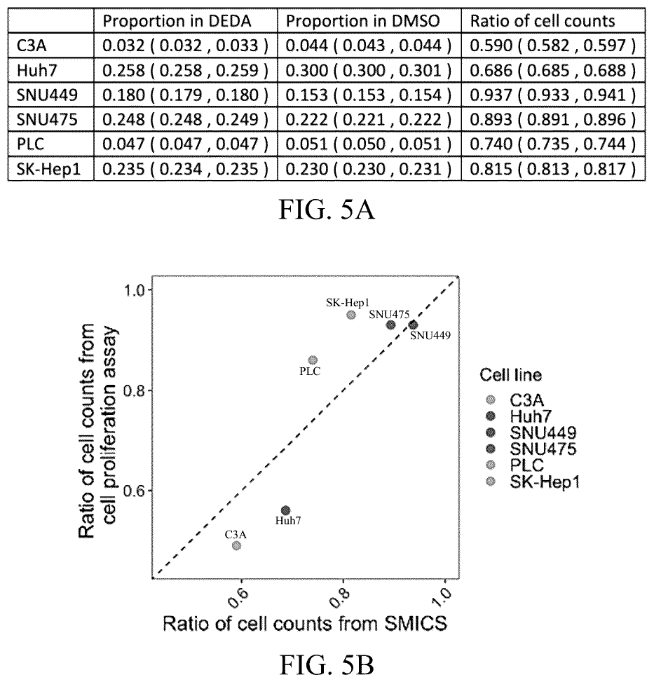

As a proof-of-concept study to validate the presently disclosed screening method, a promising halogenated diarylacetylenes, namely 4-((2,6-difluorophenyl)ethynyl)-N,N-dimethylaniline (DEDA) (FIG. 2) was tested, using six liver cancer cell lines purchased from the American Type Culture Collection (ATCC). In the preliminary experiments using the traditional drug screening method, it was found that these six cell lines have different sensitivities to DEDA. Thus, these cell lines provided excellent models to test the SMICS method. For example, DEDA inhibited c-Myc expression in C3A cells but not in SK-Hep1 cells (FIG. 3) when treated with 1 μM of DEDA or same volume of control vehicle DMSO. After 30 h, we analyzed the sensitivity of these six cell lines using a cell proliferation assay. It was found that Huh7 and C3A cells were DEDA-sensitive whereas PLC/PRF/5, SNU449, SNU475 and SK-Hep1 were resistant (FIG. 4A). The changes in the cell morphologies of individual cell lines and the mixture of six cell lines with or without drug treatment by microscope were studied (FIG. 4B).

In a parallel experiment, a mixture of six cell lines with 1 μM of DEDA or same volume of DMSO in triplicate in six-well plates were treated. After 30 h, the cells in each well were collected and the genomic DNA was isolated and analyzed by whole exome sequencing (WES) (Table 1).

TABLE 1
Summary of Statistics of Whole Exome Sequencing (WES).

| | | | | | Median |
|---|---|---|---|---|---|
| | | | | | Read-Depth |
| | Total | Total | | Uniquely | at |
| Sample | Reads | Mapped | Duplicates | Mapped | SNP loci* |
| DEDA_1 | 107369079 | 107324280 | 28748510 | 78575770 | 104 |
| DEDA_2 | 96615978 | 96548346 | 27400239 | 69148107 | 88 |
| DEDA_3 | 102959619 | 102911223 | 29110351 | 73800872 | 97 |
| DMSO_1 | 98059459 | 97962753 | 28936774 | 69025979 | 86 |
| DMSO_2 | 112334299 | 112275659 | 29218476 | 83057183 | 101 |
| DMSO_3 | 115791116 | 115711441 | 37576445 | 78134996 | 110 |

*Based on processed data of 71,488 SNPs, which were obtained from mpileup under the default parameter setting with additionally removing SNP loci having the same genotype across all six cell lines or <=30× coverage in any of the six samples. The processed data were used as the input of the CLMD algorithm to deconvolute cell line mixtures and infer drug inhibition effects.

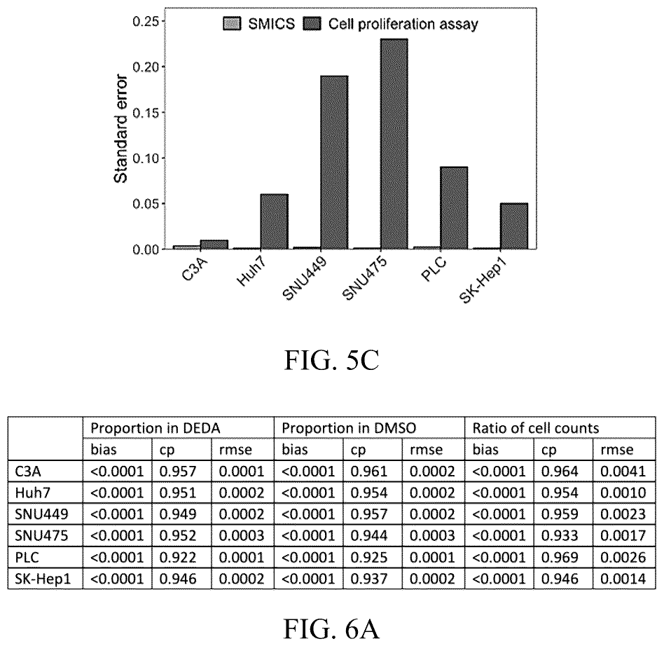

As a reference, WES of each individual cell line was also analyzed. The WES of each individual cell line identified more than 70,000 SNP sites that had polymorphisms among the six cell lines. Those sites defined a unique SNP pattern for each of the cell lines. These unique SNP patterns served as signatures of each cell line and enabled the ability to distinguish individual cell lines in the mixture samples. To facilitate this analysis, the CLMD algorithm was applied to the allele frequencies data of the SNP sites of triplicated mixture samples from DEDA or DMSO group. The proportion of each cell line in each mixture sample (FIG. 5A) was first estimated. The drug inhibition effect of each cell line was quantified by determining the ratio of cell count from DEDA-treated versus cell count in DMSO samples in which a lower ratio of cell counts indicated a stronger drug inhibition effect. The drug inhibition effect inferred from the WES of mixture samples followed the same pattern as the drug inhibition effects from each single cell line (FIG. 5B). That is, measurements of drug inhibition effects on cell lines in the mixtures were independent and consistent with drug inhibition effects on isolated cell lines in individual wells. The standard error of the estimation using mixtures was smaller than the errors obtained by using individual cell proliferation assays (FIG. 5C), an outcome suggesting a greater precision of the SMICS platform than the precision associated with individual cell lines.

### Validation Based on Simulation Studies

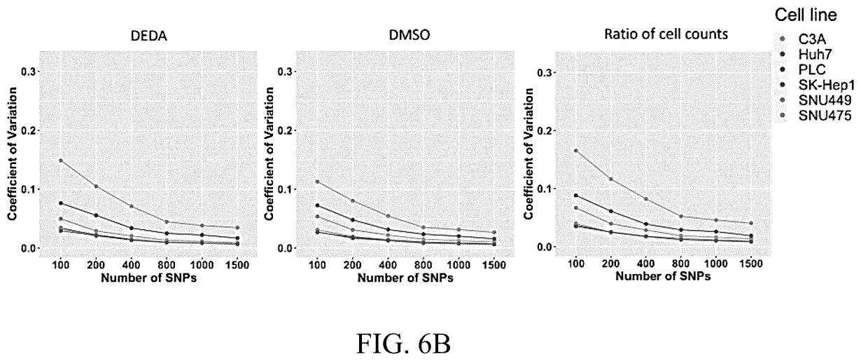

Simulation studies were performed to validate the CLMD method. The sequencing data were artificially generated based on the real data from one DEDA-treated and one DMSO sample with pre-specified mixture proportions of the six cell lines. The CLMD method was applied to estimate the mixture proportions of cell lines in each simulated sample and calculated the ratio of cell counts between DEDA-treated and DMSO samples. The simulations were replicated 1,000 times, and the results are summarized in FIG. 6A. The biases of the estimates are very small for the mixture proportions and the ratio of cell counts. The coverage probabilities (cp) of 95% confidence intervals are around the desired value of 0.95. Therefore, the CLMD method was able to estimate accurately the mixture proportions and quantify drug inhibitions.

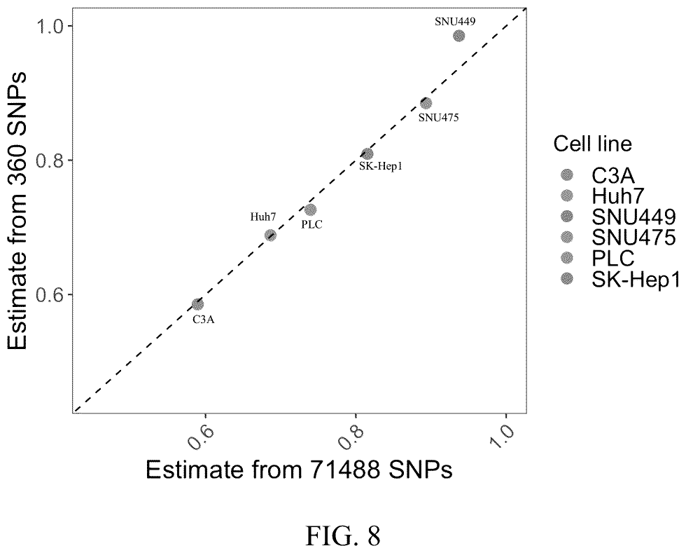

In addition, the impact of the number of SNPs on the precision of the estimation was investigated. In this regard, 50, 100, 200, 400, or 800 SNPs were randomly selected. The coefficient of variation (i.e., ratio of standard deviation/mean) of the estimates based on the selected SNPs across 1,000 simulation replicates was then calculated. For the cell line mixture proportions and ratio of cell counts (FIG. 6B) estimation, the coefficient of variations decreased as more SNPs were included in the subset. When using a subset of 400 SNPs, the coefficient of variations of the estimations of cell line proportions and ratio of cell counts were lower than 0.1 for all cell lines. This analysis suggested that instead of using WES, it was feasible to select a panel of SNPs to reduce the cost while maintaining high precision in estimating drug inhibition effects from the sample mixtures. This was further suggested by a subsequent investigation in which a panel of 360 SNPs were selected to compare with the result obtained by WES approach. As shown in FIG. 8, the drug inhibition effect assessed for the 6 cancer cell lines by the WES approach (in which all 71,488 SNPs identified for the six cancer cell lines via WES were utilized) and the selected 360 SNPs were highly consistent (R²=0.98). Such results indicate that the same outcome realized by the WES approach can be obtained via an SNP-panel approach involving significantly fewer SNPs.

### Scalability Evaluation Based on Simulation Studies

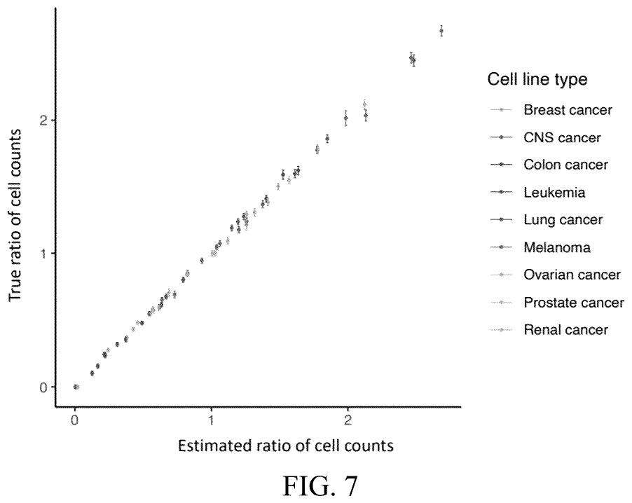

The scalability of the CLMD method to handle a mixture of NCI60 cell lines using simulation studies was assessed. A semisynthetic natural compound, called aurone-5a, was previously screened in a previous study through the NCI60 program and identified aurone-5a as a microtubule inhibitor using COMPARE.²¹ The inhibition effect reported in that study was used to set parameters in these simulations. Briefly, NGS data of a mixture of the NCI60 cell lines was simulated for six samples (three aurone-5a-treated samples and three DMSO samples), where the proportion of a cell line in an aurone-5a-treated sample was specified according to the drug inhibition effect of that cell line observed in a previous study.²¹ The CLMD method was applied to deconvolute the sample mixtures and to estimate drug inhibition effects. The estimated ratio of cell counts was consistent with the true values used to simulate the data for every single cell line (FIG. 7). The variation of the estimated values was small. This simulation indicated that this method could potentially be expanded to deconvolute a mixture of 60 cell lines and estimate drug inhibition effects.

### Discussion

A major goal of targeted therapy (i.e., “personalized medicine”) for cancer treatment seeks to identify the appropriate, effective drug for each patient's specific cancer. Combining the state-of-the-art next-generation sequencing (NGS) and advanced statistical modeling, a SNP-based mixed cell screening (SMICS) platform was developed (FIG. 1) that substantially increases the efficiency of drug screening and addresses a critical need in targeted cancer therapy. As an added advantage of this new methodology, the SMICS platform will significantly reduce the time commitment and ever-increasing cost of drug screening and thereby support modern drug development.

Drug screening in multiple cancer cell lines is a critical step in drug development and biomarker identification, and the NCI established the important NCI60 screening program to provide access to a service that was not necessarily available to investigators outside of the major pharmaceutical companies. Traditional screening, however, of individual cell lines one-by-one against a multitude of man-made, semisynthetic or naturally occurring drug candidates is exorbitantly time consuming and limited the number of different cell lines that were possible using traditional screening methods. For example, if five different concentrations of a new pharmacophore as well as a vehicle control were tested in triplicate, then each cell line would require 18 plates or wells. The expansion of this simple mathematics to the testing of hundreds of potentially interesting new compounds and multiple cell lines in triplicate reveals the time-consuming nature of this valuable but overwhelming effort, particularly for academic laboratories without access to facilities for this purpose. The PRISM methodology described herein represented a step forward to solve this limitation in traditional screening. The PRISM methodology enabled screening of mixtures of as many as 1,000 different, barcoded cell lines and correlated the cell viability in each cell line with genomic, proteomic, and metabolomic information. The barcoding procedure involved the straightforward, viral DNA insertion and stable cell selection, but introduced a potential variable in which these engineered cell lines might have different, unwanted properties relative to the original parent cell lines.

Cell lines have numerous genomic variations, including insertion-deletion mutations (INDELs) and single nucleotide polymorphisms (SNPs) that have potential as endogenous “tags” to identify a particular cell line in a mixture. As a proof-of-concept study, we developed the SMICS method using a mixture of liver cancer cell lines without virus-induced “tags” or any other genetic modifications used in the original PRISM methodology. In this study, we treated one of six individual cell lines and we treated a mixture of six cell lines with DMSO vehicle or the potential drug candidate (FIG. 4). After treatment, we isolated and analyzed the genomic DNA from the cell line mixtures using whole exome sequencing (WES). As controls, the WES of each individual liver cancer cell line was also analyzed. The WES results from triplicated samples were extremely self-consistent, and most importantly, the ratios of cell viability as determined by SMICS resembled the traditional cell screening results using each individual cell line (FIG. 5).

As evidenced, the CLMD algorithm can integrate data across all SNP sites to provide accurate estimation of the proportion of each cell line in the mixture samples, as shown in real data analysis (FIG. 5) and simulation studies (FIG. 6). It still performs well under the more complex situation of a mixture of 60 cell lines with diverse cancer types based on simulation studies that mimic real data (FIG. 7).

The CLMD algorithm estimates cell line proportions based on the maximum likelihood method, which requires numerically finding the maximization point of the likelihood function. This optimization problem is non-trivial because it is a complex constrained optimization, where all the proportions need to be between 0 and 1 and the summation of them needs to be equal to one. As described above with reference to the materials and methods, the K proportion parameters are reparamatized into K−1 independent parameters with a domain of (−∞, ∞). Therefore, the constrained optimization problem is transformed into a much simpler unconstrained optimization problem, which greatly facilitates obtaining stable estimates of the parameters. Another technical point of the CLMD algorithm is that it uses a formula-based approach to calculate the variance of the estimators. A frequently used method to calculate the variance is bootstrap. However, that method is time consuming because it requires data re-sampling. To address this issue, a variance formula was derived based on the delta method and asymptotic properties of maximum likelihood estimator. With the formula, the variance can be calculated very quickly.

Based on the data from all of the detected SNPs from WES in this analysis, the cell line proportions from sample mixtures were deconvoluted. Simulation studies (FIG. 6B) showed that a random subset of 400 randomly selected SNPs yielded sufficient accuracy in cell line proportion estimation. Simulation studies further showed that a random subset of 360 randomly selected SNPs also yielded sufficient accuracy in cell line proportion estimation (FIG. 8). That is, rather than using all SNPs, a small number of SNPs can potentially be used to discriminate different cell lines. Reducing the number of SNPs is consistent with literature information indicating that a low number of SNPs are sufficient to discriminate among different individuals as, for example, in a 24-SNP panel that possessed high discriminatory power.

Although the studies underlying the current Example only utilized six cell lines, the results suggested it is feasible to perform large-scale screening using a mixture of multiple “intact” cell lines without introducing extra “tags”. For example, we performed a simulation study using the NCI60 cell lines, which demonstrated that this method could potentially expand to deconvolute a mixture of 60 cell lines and estimate drug inhibition effects. Finally, the algorithm developed in this study could be used to analyze cell mixtures in vivo as well. In summary, SMICS provides a methodology which may significantly improve the efficiency of drug discovery by reducing the time commitment and cost of drug screening and biomarker identification.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1 Shoemaker, R. H. The NCI60 human tumour cell line anticancer drug screen. Nat Rev Cancer 6, 813-823, doi: 10.1038/nrc 1951 (2006).
2 Paull, K. D. et al. Display and analysis of patterns of differential activity of drugs against human tumor cell lines: development of mean graph and COMPARE algorithm. J Natl Cancer Inst 81, 1088-1092, doi: 10.1093/jnci/81.14.1088 (1989).
3 Barretina, J. et al. The Cancer Cell Line Encyclopedia enables predictive modelling of anticancer drug sensitivity. Nature 483, 603-607, doi: 10.1038/nature11003 (2012).
4 Garnett, M. J. et al. Systematic identification of genomic markers of drug sensitivity in cancer cells. Nature 483, 570-575, doi: 10.1038/nature11005 (2012).
5 Greshock, J. et al. Molecular target class is predictive of in vitro response profile. Cancer Res 70, 3677-3686, doi: 10.1158/0008-5472.CAN-09-3788 (2010).
6 Haverty, P. M. et al. Reproducible pharmacogenomic profiling of cancer cell line panels. Nature 533, 333-337, doi: 10.1038/nature17987 (2016).
7 Yu, C. et al. High-throughput identification of genotype-specific cancer vulnerabilities in mixtures of barcoded tumor cell lines. Nat Biotechnol 34, 419-423, doi: 10.1038/nbt.3460 (2016).
8 Jin, X. et al. A metastasis map of human cancer cell lines. Nature 588, 331-336, doi: 10.1038/s41586-020-2969-2 (2020).
9 Li, H. et al. The landscape of cancer cell line metabolism. Nat Med 25, 850-860, doi: 10.1038/s41591-019-0404-8 (2019).
10 Rees, M. G. et al. Systematic identification of biomarker-driven drug combinations to overcome resistance. Nat Chem Biol 18, 615-624, doi: 10.1038/s41589-022-00996-7 (2022).
11 Pengelly, R. J. et al. A SNP profiling panel for sample tracking in whole-exome sequencing studies. Genome medicine 5, 1-7 (2013).
12 Pakstis, A. J. et al. SNPs for a universal individual identification panel. Human genetics 127, 315-324 (2010).
13 Yousefi, S. et al. A SNP panel for identification of DNA and RNA specimens. BMC genomics 19, 1-12 (2018).
14 Kidd, K. K. et al. Developing a SNP panel for forensic identification of individuals. Forensic science international 164, 20-32 (2006).
15 Novroski, N. M. & Cihlar, J. C. Evolution of single-nucleotide polymorphism use in forensic genetics. Wiley Interdisciplinary Reviews: Forensic Science 4, e1459 (2022).
16 Desai, J. R., Ochoa, S., Prins, P. A. & He, A. R. Systemic therapy for advanced hepatocellular carcinoma: an update. J Gastrointest Oncol 8, 243-255, doi: 10.21037/jgo.2017.02.01 (2017).
17 Doycheva, I. & Thuluvath, P. J. Systemic Therapy for Advanced Hepatocellular Carcinoma: An Update of a Rapidly Evolving Field. J Clin Exp Hepatol 9, 588-596, doi: 10.1016/j.jceh.2019.07.012 (2019).
18 Sviripa, V. M. et al. Phenylethynyl-substituted Heterocycles Inhibit Cyclin D1 and Induce the Expression of Cyclin-dependent Kinase Inhibitor p21 (Wif1/Cip1) in Colorectal Cancer Cells. Medchemcomm 9, 87-99, doi: 10.1039/C7MD00393E (2018).
19 Sviripa, V. M. et al. Halogenated diarylacetylenes repress c-myc expression in cancer cells. Bioorg Med Chem Lett 24, 3638-3640, doi: 10.1016/j.bmcl.2014.04.113 (2014).
20 Murray, B., Barbier-Torres, L., Fan, W., Mato, J. M. & Lu, S. C. Methionine adenosyltransferases in liver cancer. World J Gastroenterol 25, 4300-4319, doi: 10.3748/wjg.v25.i31.4300 (2019).
21 Xie, Y. et al. Semisynthetic aurones inhibit tubulin polymerization at the colchicine-binding site and repress PC-3 tumor xenografts in nude mice and myc-induced T-ALL in zebrafish. Sci Rep 9, 6439, doi: 10.1038/s41598-019-42917-0 (2019).
22 McFarland, J. M. et al. Multiplexed single-cell transcriptional response profiling to define cancer vulnerabilities and therapeutic mechanism of action. Nat Commun 11, 4296, doi: 10.1038/s41467-020-17440-w (2020).
23 Tsvetkov, P. et al. Mitochondrial metabolism promotes adaptation to proteotoxic stress. Nat Chem Biol 15, 681-689, doi: 10.1038/s41589-019-0291-9 (2019).
24 Bolger, A. M., Lohse, M. & Usadel, B. Trimmomatic: a flexible trimmer for Illumina sequence data. Bioinformatics 30, 2114-2120 (2014).
25 Institute, B. Picard Tools, <http://broadinstitute.github.io/picard/> (2016).
26 DePristo, M. A. et al. A framework for variation discovery and genotyping using next-generation DNA sequencing data. Nature genetics 43, 491-498 (2011).
27 Li, H. A statistical framework for SNP calling, mutation discovery, association mapping and population genetical parameter estimation from sequencing data. Bioinformatics 27, 2987-2993 (2011).
28 Reinhold, W. C. et al. CellMiner: a web-based suite of genomic and pharmacologic tools to explore transcript and drug patterns in the NCI-60 cell line set. Cancer research 72, 3499-3511 (2012).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of screening a test compound against multiple cell lines, comprising:
(a) treating a mixture with the test compound;
(b) identifying allele frequencies of single nucleotide polymorphisms (SNPs) in the treated mixture and in a control mixture, wherein the treated mixture and the control mixture each include cells from each of the multiple cell lines that are free of exogenous barcode nucleotides, and wherein each cell line of the multiple cell lines have one or more unique patterns of SNPs relative to the other cell lines of the multiple cell lines;
(c) estimating a proportion of each of the multiple cell lines in the treated mixture and a proportion of each of the multiple cell lines in the control mixture via one or more processors executing instructions stored in memory to perform deconvolution of the treated mixture and the control mixture based, at least in part, on
(i) a comparison of the identified allele frequencies of SNPs in the treated mixture and in the control mixture to a predetermined number of SNPs, and
(ii) the one or more unique patterns of SNPs associated with each of the multiple cell lines in the treated mixture and the control mixture; and
(d) quantifying the effect of the test compound on each of the multiple cell lines based on the estimated proportion of each of the multiple cell lines in the treated mixture and the estimated proportion of each of the multiple cell lines in the control mixture.

2. The method as recited in claim 1, wherein next generation sequencing (NGS) is utilized in the identification of the allele frequencies of SNPs in the treated mixture and the control mixture.

3. The method as recited in claim 1, wherein quantifying the effect of the test compound on each of the multiple cell lines includes generating a value for each cell line of the multiple cell lines indicative of the inhibitory effect of the test compound on the cell line.

4. The method as recited in claim 1, wherein the predetermined number of SNPs to which the identified allele frequencies of SNPs for the treated mixture and the control mixture are compared is equal to a total number of SNPs identified across the multiple cell lines.

5. The method as recited in claim 1, wherein the predetermined number of SNPs to which the identified allele frequencies of SNPs for the treated mixture and the control mixture are compared is equal to a subset of a total number of SNPs identified across the multiple lines.

6. The method as recited in claim 5, wherein the subset of the total number of SNPs comprises 400 or fewer SNPs.

7. The method as recited in claim 1, wherein the control mixture is treated with a control compound.

8. The method as recited in claim 7, wherein the control compound is dimethylsulfoxide (DMSO).

9. The method as recited in claim 1, wherein each cell line of the multiple cell lines is a cancer cell line.

10. The method as recited in claim 1, wherein each cell line of the multiple cell lines is a liver cancer cell line.

11. The method of claim 1, wherein the multiple cell lines comprises one or more cell lines selected from the group consisting of: Huh7, C3A, PLC/PRF/5, SNU449, and SK-Hep1.

12. The method of claim 1, wherein the multiple cell lines comprises at least six cell lines.

13. A system for screening a test compound against multiple cell lines, comprising:
one or more processors; and
memory storing instructions that, when executed by the one or more processors, cause the one or more processors to perform operations that include
(a) comparing data corresponding to allele frequencies of single nucleotide polymorphisms (SNPs) in a first mixture treated with the test compound to a predetermined number of SNPs, wherein the first mixture include cells from each of the multiple cell lines that are free of exogenous barcode nucleotides;
(b) comparing data corresponding to allele frequencies of SNPs in a second mixture to the predetermined number of SNPs, wherein the second mixture include cells from each of the multiple cell lines that are free of exogenous barcode nucleotides;
(c) estimating a proportion of each of the multiple cell lines in the first mixture by performing deconvolution of the first mixture based, at least in part, on
(i) the comparison of the data corresponding to allele frequencies of SNPs in the first mixture to the predetermined number of SNPs, and
(ii) one or more unique patterns of SNPs associated with each respective cell line of the multiple cell lines;
(d) estimating a proportion of each of the multiple cell lines in the second mixture by performing deconvolution of the second mixture based, at least in part, on
(i) the comparison of the data corresponding to allele frequencies of SNPs in the second mixture to the predetermined number of SNPs, and
(ii) the one or more unique patterns of SNPs associated with each respective cell line of the multiple cell lines; and
(e) quantifying the effect of the test compound on each of the multiple cell lines based on the estimated proportion of each of the multiple cell lines in the first mixture and the estimated proportion of each of the multiple cell lines in the second mixture.

14. The system as recited in claim 13, wherein quantifying the effect of the test compound on each of the multiple cell lines includes generating a value for each cell line of the multiple cell lines indicative of the inhibitory effect of the test compound on the cell line.

15. The system as recited in claim 13, wherein the predetermined number of SNPs is equal to a total number of SNPs identified across the multiple cell lines.

16. The system as recited in claim 13, wherein the predetermined number of SNPs is equal to a subset of a total number of SNPs identified across the multiple cell lines.

17. The system as recited in claim 16, wherein the subset of the total number of SNPs comprises 400 or fewer SNPs.

18. The system as recited in claim 13, wherein each cell line of the multiple cell lines is a cancer cell line.

19. The system as recited in claim 13, wherein the multiple cell lines comprises at least six cell lines.

20. A system for identifying a proportion of a particular cell line in a mixture including multiple cell lines, comprising:
one or more processors; and
memory storing instructions that, when executed by the one or more processors, cause the one or more processors to perform operations that include
(a) comparing data corresponding to allele frequencies of single nucleotide polymorphisms (SNPs) in the mixture to a predetermined number of SNPs, wherein the mixture includes cells from each of the multiple cell lines that are free of exogenous barcode nucleotides; and
(b) estimating the proportion of the particular cell line in the mixture by performing deconvolution of the mixture based on
(i) the comparison of the data corresponding to the allele frequencies of SNPs in the mixture to the predetermined number of SNPs, and
(ii) one or more unique patterns of SNPs associated with each respective cell line of the multiple cell lines.
